(19) United States
(12) Patent Application Publication
(10) Pub. No.: US 2022/0073648 A1
(43) Pub. Date: Mar. 10, 2022

(54) INFLUENZA VACCINES AND METHODS OF USE THEREOF

(71) Applicant: Stephen D. Gillies, Carlisle, MA (US)

(72) Inventor: Stephen D. Gillies, Carlisle, MA (US)

(21) Appl. No.: 17/235,895

(22) Filed: Apr. 20, 2021

Related U.S. Application Data
(63) Continuation of application No. 15/320,679, filed on Dec. 20, 2016, now abandoned.
(60) Provisional application No. 62/015,124, filed on Jun. 20, 2014.

(51) Int. Cl. C07K 16/42; C07K 16/10; A61K 39/12; A61K 38/19; A61K 39/395; C07K 14/535

(52) U.S. Cl. CPC C07K 16/4216; C07K 16/1018; A61K 39/12; A61K 38/193; A61K 2039/505; C07K 14/535; C07K 2317/622; C07K 2317/21; C07K 2317/76; A61K 39/39566

(57) ABSTRACT

The disclosure relates to anti-idiotypic antibodies and related influenza virus vaccines.

### CROSS-REFERENCED TO RELATED APPLICATIONS

This application claims the benefit under 35 U.S.C. § 119(e) of U.S. provisional application No. 62/015,124, filed Jun. 20, 2014, which is incorporated by reference herein in its entirety.

### BACKGROUND OF THE DISCLOSURE

Influenza viruses that routinely spread in humans are responsible for seasonal flu epidemics each year. Currently annual influenza vaccinations are used to protect against the influenza virus. Each seasonal influenza vaccine typically contains antigens of multiple influenza virus strains, e.g., one influenza type A subtype H1N1 virus strain, one influenza type A subtype H3N2 virus strain, and either one or two influenza type B virus strains. Influenza vaccines may be administered as an injection, also known as a flu shot, or as a nasal spray.

### SUMMARY OF THE DISCLOSURE

Aspects of the disclosure relate to compositions and methods for treating infectious disease caused by RNA viruses of the family Orthomyxoviridae, the influenza viruses. In some embodiments, a vaccine is provided to treat seasonal and/or pandemic flu. In some embodiments, a vaccine comprises an anti-idiotypic antibody comprising an idiotope that mimics or resembles an immunogenic region of an influenza virus (e.g., of a surface protein of an influenza virus). In some embodiments, the immunogenic region can be of any influenza virus strain (e.g., H1N1, H5N1) of any antigen type (e.g., A, B, C) known in the art. In some embodiments, a vaccine comprises an anti-idiotypic antibody comprising an idiotope that mimics or resembles a fusion domain or a portion thereof of a stalk region of a hemagglutinin protein of an influenza virus. Aspects of the disclosure provide anti-idiotypic antibodies comprising one or more idiotopes mimicking one or more influenza virus antigens.

These and other aspects of the disclosure are further illustrated by the following description and examples.

### BRIEF DESCRIPTION OF THE DRAWINGS

FIG. 1A is a non-limiting illustration of an influenza virion;

FIG. 1B is a non-limiting illustration of a hemagglutinin (HA) protein of an influenza virus, in which is depicted the receptor binding pocket and fusion peptide portions;

FIG. 2 illustrates a non-limiting embodiment of an anti-idiotypic vaccine production process;

FIG. 3 is a non-limiting illustration of results of an ELISA assay showing inhibition, by various anti-idiotypic antibodies, of HA protein binding to an antibody (referred to as scFv-F10-mγ2a antibody (PR1)) that targets the stalk region of HA protein;

FIG. 4 illustrates a non-limiting illustration of results of an SDS-PAGE assay showing a purified chimeric anti-idiotypic antibody (referred to as c7G7) under reducing (R) and non-reducing (NR) conditions;

FIG. 5 is a non-limiting illustration of results of an ELISA assay showing binding of c7G7 to PR1;

FIG. 6 illustrates a non-limiting embodiment of results of an ELISA assay showing inhibition of binding HA protein to PR1;

FIG. 7 is a non-limiting illustration of a rabbit immunization protocol;

FIG. 8 illustrates a non-limiting embodiment of analysis of response of c7G7 immune rabbit sera to c7G7 that contains human C regions;

FIG. 9 illustrates a non-limiting embodiment of analysis of response of c7G7 immune rabbit sera to the original mouse 7G7 (which may be referred to herein as m7G7) that shares mouse V regions with c7G7;

FIG. 10 illustrates a non-limiting embodiment of analysis of response of c7G7 immune rabbit sera that blocks the interaction between c7G7 and PR1;

FIG. 11 illustrates a non-limiting embodiment of analysis of response of c7G7 immune rabbit sera to H5N1 HA protein;

FIG. 12 illustrates a non-limiting embodiment of analysis of 7G7 immune mouse sera;

FIG. 13 illustrates a non-limiting embodiment of a mouse model vaccine construct;

FIGS. 14A-14B illustrate non-limiting embodiments of an enzyme-linked immunosorbent assay for evaluating binding to hemagglutinin protein of an influenza virus expressed in transiently transfected mammalian cells;

FIG. 15A-15D illustrate non-limiting embodiments of a syncytia inhibition assay;

FIG. 16 illustrates non-limiting embodiments of potential immunogenic portions of an anti-idiotypic antibody in humans; and

FIGS. 17A-17B illustrate non-limiting embodiments of cleavage sites in hemaglutinin proteins.

### DETAILED DESCRIPTION OF THE DISCLOSURE

Aspects of the disclosure relate to anti-idiotypic antibodies and related vaccines. In some embodiments, vaccines are provided that comprise anti-idiotypic antibodies that have immunogenic regions useful for inducing an immune response (e.g., a cellular and/or humoral immune response) against an infectious agent (e.g., an influenza virus). In some embodiments, anti-idiotypic antibodies have immunogenic regions useful for inducing an immune response that is effective against a broad spectrum of seasonal and/or pandemic influenza viruses, e.g., influenza viruses of the Type A, B and/or C.

In some embodiments, anti-idiotypic antibodies of the disclosure are useful because they are directed against idiotopes of antibodies that target relatively invariant regions of influenza virus (e.g., the stalk region of a hemagglutinin protein that controls cell fusion) such that they make it more difficult for a virus to escape immune surveillance by antigenic variation. In some embodiments, anti-idiotypic antibodies of the disclosure are directed against idiotopes of antibodies that target a subregion of hemagglutinin corresponding to peptide(s) that mediate cell fusion. In some embodiments, anti-idiotypic antibodies of the disclosure are useful because they obviate the need to obtain large quantities of inactivated virus product for vaccine production. In some embodiments, anti-idiotypic antibodies of the disclosure are useful because they are effective for inducing an immune response in a subject, e.g., an infant, that is incapable or has a limited ability to respond effectively to a viral antigen per se.

In some embodiments, anti-idiotypic antibodies comprise idiotopes having immunogenic regions that mimic or resemble an immunogenic region of an infectious agent (e.g., an influenza virus). An immunogenic region may be a three-dimensional epitope (e.g., formed by a secondary or tertiary protein structure) of the infectious agent. However, in some embodiments, an immunogenic region may be a linear sequence of amino acids. Thus, in some embodiments, anti-idiotypic antibodies carry an internal image of the original antigen of the infectious agent. In some embodiments, anti-idiotypic antibodies, which mimic or resemble an immunogenic region of an infectious agent, are capable of producing an immune response (cellular and/or humoral) in a subject at levels comparable to an infectious agent. In some embodiments, anti-idiotypic antibodies mimic or resemble an epitope of a stalk region (e.g., a fusion peptide epitope (which mediates cell fusion)) of an influenza virus hemagglutinin protein.

In some embodiments, the extent of a humoral response may be determined by using an appropriate immunoassay, such as an ELISA, a radio-immuno assay or other specific binding assay (e.g. surface plasmon resonance) using sera from a vaccinated subject. In some embodiments, the extent of a cellular response can be determined using any appropriate assay, including, for example, a T-cell activation assay, an IFN-gamma production assay or a cytokine ELISPOT or intracellular expression assay.

In some embodiments, anti-idiotypic antibodies, which mimic or resemble an immunogenic region of an infectious agent, compete with the infectious agent for binding to a ligand of the infectious agent (e.g., a cell surface receptor, an antibody or antibody binding fragment that specifically binds to the infectious agent).

In some embodiments, the extent to which an anti-idiotypic antibody can mimic an antigen (e.g., an infectious agent or its specific protein (in purified form or expressed on a cell surface)) by eliciting an effective anti-anti-immune response is tested first by determining how strongly it binds to Ab1, which is the antibody having the idiotope against which the anti-idiotypic antibody is directed. In some embodiments, the anti-idiotypic antibody binds to Ab1 with a binding affinity (K_{D}) in the range of 0.01 nM to 100 nM, 0.1 nM to 10 nM, or 0.1 nM to 3 nM. In some embodiments, the anti-idiotypic antibody binds to Ab1 with a binding affinity (K_{D}) of less than 1 μM, less than 1 nM, or less than 1 μM. In some embodiments, the anti-idiotypic antibody binds to Ab1 with a binding affinity (K_{D}) of about 10⁻⁷ M, about 10⁻⁸ M, about 10⁻⁹ M, about 10⁻¹⁰ M, about 10⁻¹¹ M about 10⁻¹² M, or about 10⁻¹³ M.

In some embodiments, the anti-idiotypic antibody binds to Ab1 with a binding affinity that is in a range of 0.1 times to 5 times, 0.5 times to 1.5 times, 1 times to 2.5 times, or 1 times to 5 times the binding affinity of the antigen to Ab1. In some embodiments, the anti-idiotypic antibody binds to Ab1 with a binding affinity that is about 0.1 times, 0.2 times, about 0.3 times, about 0.4 times, about 0.5 times, about 0.6 times, about 0.7 times, about 0.8 times, about 0.9 times, about 1 times, about 1.1 times, about 1.2 times, about 1.3 times, about 1.4 times, about 1.5 times, about 1.6 times, about 1.6 times, about 1.7 times, about 1.8 times, about 1.9 times, about 2 times, about 2.5 times, about 5 times the binding affinity of the antigen to Ab1 or more.

In some embodiments, binding of an anti-idiotypic antibody to Ab1 prevents Ab1 from binding to its antigen. Thus, in some embodiments, the extent to which an anti-idiotypic antibody can mimic an antigen can be determined by evaluating the extent to which the anti-idiotypic antibody can prevent Ab1 from binding to its antigen. In some embodiments, in reference to competition with antigen for binding to Ab1, the anti-idiotypic antibody has an inhibitory constant (Kᵢ) in the range of 0.01 nM to 100 nM, 0.1 nM to 10 nM, or 0.1 nM to 3 nM. In some embodiments, in reference to competition with antigen for binding to Ab1, the anti-idiotypic antibody has an inhibitory constant (Kᵢ) of less than 1 μM, less than 1 nM, or less than 1 μM. In some embodiments, in reference to competition with antigen for binding to Ab1, the anti-idiotypic antibody has an inhibitory constant (Kᵢ) about 10⁻⁷ M about 10⁻⁸ M, about 10⁻⁹ M, about 10⁻¹⁰ M, about 10⁻¹¹ M, about 10⁻¹² M, or about 10⁻¹³ M.

In some embodiments, in reference to competition with antigen for binding to Ab1, the anti-idiotypic antibody has an half maximal inhibitory concentration (IC50) in the range of 0.01 nM-100 nM, 0.1 nM-10 nM, or 0.1 nM to 3 nM. In some embodiments, in reference to competition with antigen for binding to Ab1, the anti-idiotypic antibody has an IC50 of less than 1 μM, less than 1 nM, or less than 1 μM. In some embodiments, in reference to competition with antigen for binding to Ab1, the anti-idiotypic antibody has an IC50 about 10⁻⁷ M, about 10⁻⁸ M, about 10⁻⁹ M, about 10⁻¹⁰ M, about 10⁻¹¹ M about 10⁻¹² M or about 10⁻¹³ M.

In some embodiments, the anti-idiotypic antibody is capable of producing an immune response in an animal (e.g., a rabbit, a mouse, a human) that results in the production of anti-anti-idiotypic antibodies that bind to the original antigen (against which Ab1 was raised) with a binding affinity (K_{D}) in the range of 0.01 nM to 100 nM, 0.1 nM to 10 nM, or 0.1 nM to 3 nM. In some embodiments, the anti-idiotypic antibody is capable of producing an immune response in an animal (e.g., a rabbit, a mouse, a human) that results in the production of anti-anti-idiotypic antibodies that bind to the original antigen (against which Ab1 was raised) with a binding affinity (K_{D}) of less than 1 μM, less than 1 nM, or less than 1 pM.

In some embodiments, anti-idiotypic antibodies comprise idiotopes having immunogenic regions that mimic an immunogenic region of viral surface or coat protein. In some embodiments, idiotopes comprise immunogenic regions that mimic a three-dimensional immunogenic region of viral surface or coat protein of an influenza virus. In some embodiments, idiotopes comprise immunogenic regions that mimic an immunogenic region of viral surface or coat protein of an influenza virus of the Type A, B and/or C.

Influenza viruses may be subclassified by their two major surface proteins: hemagglutinin and neuraminidase.

In some embodiments, anti-idiotypic antibodies comprise idiotopes that mimic a three-dimensional immunogenic region of hemagglutinin. Hemagglutinin mediates viral cell entry in part by recognizing host proteins bearing sialic acids on their surface and triggering fusion of viral and host membranes allowing viral RNA to enter the cytoplasm via endocytosis. Accordingly, in some embodiments, anti-idiotypic antibodies are provided that are effective for inducing an immune response that comprises production of antibodies that specifically bind to hemagglutinin and block cell entry, thereby neutralizing the virus. Aspects of the disclosure provide anti-idiotypic antibodies that induce immune response in a subject that comprises production of antibodies that bind specifically to a highly conserved epitope within the stalk region of hemagglutinin. In some embodiments, such antibodies inhibit the post-attachment fusion process. In some embodiments, such antibodies inhibit the post-attachment fusion process and viral entry into cells, but not binding to the cell surface.

In some embodiments, anti-idiotypic antibodies are provided that induce immune response in a subject that comprises production of antibodies that bind specifically to one or more of hemagglutinin subtypes H1-16 of influenza A viruses. In some embodiments, an influenza virus is selected from the group consisting of H5N1, H1N1, H2N2, H6N1, H6N2, H8N4, H9N2 and H3N2 influenza viruses. In some embodiments, an influenza virus is selected from the group consisting of the following influenza strains: H1-OH83 (A/Ohio/83 (H1N1)); H1-PR34 (A/Puerto Rico/8/34 (H1N1)); H1-SC1918 (A/South Carolina/1/1918 (H1N1)); H1-WSN33 (A/WSN/1933 (H1N1)); H2-AA60 (A/Ann Arbor/6/60 (H2N2)); H2-JP57 (A/Japan/305/57(H2N2)); H3-SY97 (A/Sydney/5/97(H3N2)); H6-HK99 (A/quail/Hong Kong/1721-30/99(H6N1)); H6-NY98 (A/chicken/New York/14677-13/1998 (H6N2)); H7-FP34 (A/FPV/Rostock/34 (H7N1)); H8-ON68 (A/turkey/Ontario/6118/68); H9-HK(G9)97 (A/chicken/HongKong/G9/97 (H9N2)); H9-HK99 (A/HongKong/1073/99 (H9N2)); H11-MP74 (A/duck/Memphis/546/74 (H11N9)); and H7N9 (A/Shanghai/2/2013).

Non-limiting examples include influenza viruses described in Damian C. Ekiert and Ian A. Wilson, Broadly neutralizing antibodies against influenza virus and prospects for universal therapies, Curr Opin Virol. 2012 April; 2(2): 134-141; Jianhua Sui, et al., Structural and functional bases for broad-spectrum neutralization of avian and human influenza A viruses, Nat Struc Mol Bio (published online 22 Feb. 2009) pages 1-9; and Han Zhang, et al., Universal Influenza Vaccines, a Dream to Be Realized Soon Viruses. Viruses (2014) 6, 1974-1991.

While aspects of the disclosure relate to anti-idiotypic antibodies mimicking immunogenic regions of hemagglutinin, in some embodiments, anti-idiotypic antibodies are provided that comprise idiotopes that mimic or resemble immunogenic regions of other antigens, such as, for example, neuraminidase (e.g., one or more of neuraminidase subtypes N1-9). Neuraminidase is an enzyme that cleaves sialic acid from host and viral proteins, facilitating cell exit. Accordingly, in some embodiments, anti-idiotypic antibodies are provided that are effective for inducing an immune response in a subject that comprises production of antibodies that specifically bind to neuraminidase and block cell exit.

### Antibodies

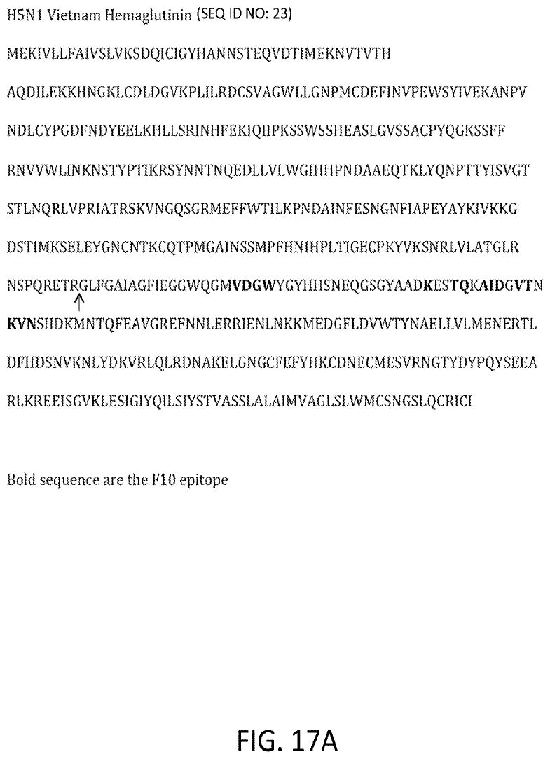

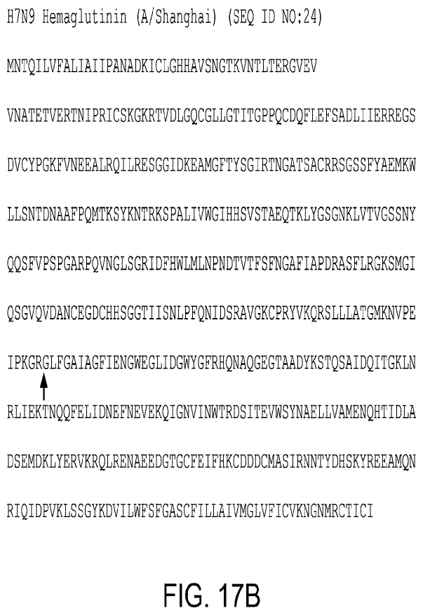

In some embodiments, an anti-idiotypic antibody is provided that mimics an immunogenic region of one or more influenza viruses. In some embodiments, an anti-idiotypic antibody is provided that mimics an immunogenic region of hemagglutinin of an influenza virus. In some embodiments, an anti-idiotypic antibody is provided that mimics a fusion domain of a stalk region of hemagglutinin. In some embodiments, this region is adjacent (in the carboxyl terminal direction) to the proteolytic cleavage site that generates a new N-terminus that inserts in the membrane at the low pH of the endosome. Examples of cleavage sites in H7N9 Hemaglutinin (A/Shanghai) and H5N1 Vietnam Hemaglutinin are shown in FIGS. 17A (SEQ ID NO: 23) and 17B (SEQ ID NO: 24). An example amino acid sequence of H5N1 Vietnam Hemaglutinin is also provided at SEQ ID NO: 25.

In some embodiments, an anti-idiotypic antibody provided herein is useful as a vaccine against flu because it brings about production of a broad-spectrum antibody and/or cellular response. In some embodiments, an anti-idiotypic antibody provided herein is useful as a vaccine against flu because the stalk region is more conserved and does not mutate as fast as the head region (e.g., receptor binding pocket) of the HA protein.

Anti-idiotypic antibodies can be of any suitable antibody type. The term “antibody” as used herein refers to immunoglobulin molecules and immunologically active portions of immunoglobulin molecules, e.g., molecules that contain an antigen binding site that specifically binds (immunoreacts with) an antigen and/or that mimic or resemble an immunogenic antigen of interest. The term also encompasses any molecule having a binding domain which is homologous or largely homologous to an immunoglobulin binding domain. An antibody may be monoclonal or polyclonal. An antibody may be a member of any immunoglobulin class, including any of the human classes: IgG, IgM, IgA, IgD, and IgE. Derivatives of the IgG class, for example, include IgG1, IgG2, IgG3, and IgG4.

The term antibody encompass immunoglobulin fragments, such as, for example, Fab, Fab′, F(ab′)2, scFv, Fv, dsFv diabody, and Fd fragments.

Single-chain Fvs (scFvs) comprise the variable light chain (VL) and variable heavy chain (VH) covalently connected to one another by a polypeptide linker. Either VL or VH may be the NH2-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility.

Diabodies are dimeric scFvs. The components of diabodies typically have shorter peptide linkers than most scFvs, and they show a preference for associating as dimers.

An Fv fragment comprises one VH and one VL domain held together by noncovalent interactions. The term dsFv is used herein to refer to an Fv with an engineered intermolecular disulfide bond to stabilize the VH-VL pair.

A F(ab′)2 fragment is an antibody obtained from immunoglobulins (e.g., an IgG) by digestion with an appropriate enzyme, such as pepsin at pH 4.0-4.5. Such fragments may also be recombinantly produced.

A Fab fragment is an antibody obtained by reduction of the disulfide bridge or bridges joining the two heavy chain pieces in the F(ab′)2 fragment. Such fragments may also be recombinantly produced. In some embodiments, a Fab fragment is an antibody obtained by digestion of immunoglobulins (typically IgG) with the enzyme papain. A Fab fragment may be recombinantly produced. The heavy chain segment of the Fab fragment is the Fd fragment.

In some embodiments, anti-idiotypic antibodies are in the form of single chain antibodies (including but not limited to scFvs). In some embodiments, anti-idiotypic antibodies are in the form of nanoantibodies. In some embodiments, nanoantibodies are single-domain VHH antibodies derived from camelidae (camels, llamas, alpacas, etc.).

It also should be appreciated that antibodies can be chimeric (e.g., portions from different species, different subtypes, etc.) and/or modified (e.g., humanized) to alter their activity and/or immunogenicity in a recipient organism. Antibodies may also be conjugation to carriers and/or adjuvants. Furthermore, methods for enhancing immune responses to an anti-idiotypic antibody may include the use of adjuvants. In some embodiments, an anti-idiotypic antibody may be attached to a cytokine. Preferred cytokines include interleukins such as interleukin-2 (IL-2), IL-4, IL-7, IL-12, IL-15, IL-18, IL-21, and IL-23, as well as factors such as granulocyte-macrophage colony stimulating factor (GM-CSF), tumor necrosis factors (TNF) such as TNFα, lymphokines such as lymphotoxin, and interferons such as interferon α, interferon β, and interferon γ, and chemokines.

The specificity of an anti-idiotypic antibody can be evaluated using techniques known in the art (e.g., as illustrated in the Examples). As used herein, the term “binds specifically” means that the antibody is capable of specific binding to its target antigen in the presence of the antigen under suitable binding conditions known to one of skill in the art. In some embodiments, the antibody has an affinity constant, Kₐ in a range of 10⁷ M⁻¹ to 10⁸ M⁻¹, 10⁸ M⁻¹ to 10⁹ M⁻¹, 10⁹ M⁻¹ to 10¹⁰ M⁻¹, 10¹⁰ M⁻¹ to 10¹¹ M⁻¹, or 10¹¹ M⁻¹ to 10¹² M⁻¹. In some embodiments, the antibody or recombinant antibody has an affinity constant, Kₐ of at least 10⁷ M⁻¹, at least 10⁸M⁻¹, at least 10⁹ M⁻¹, at least 10¹⁰ M⁻¹, at least 10¹¹ M⁻¹, or at least 10¹² M⁻¹.

In some embodiments, “binds specifically” means that at least 90 percent, at least 95 percent, at least 98 percent, or at least 99 percent, of antibody-antigen immune complexes formed when the antibody is contacted with a source of antigens, under conditions suitable for the formation of immune complexes, include a specified antigen.

### Antibody Production

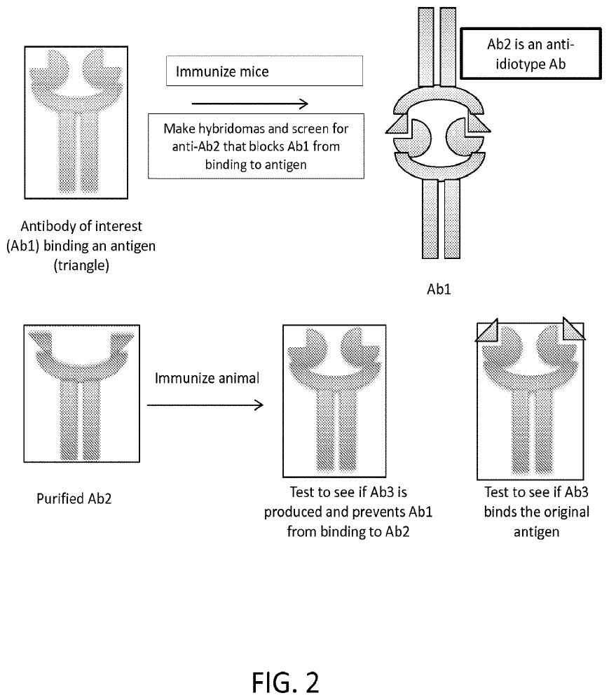

Methods of producing an anti-idiotypic antibody are known in the art (for example in H. Koprowski Unconventional Vaccines: Immunization with Anti-Idiotype Antibody against Viral Diseases Cancer Res 1985; 45:4689s-4690s.). In some embodiments, an anti-idiotypic antibody can be made as depicted in FIG. 2. In some embodiments, an anti-idiotypic antibody can be made by:
obtaining (e.g., producing or identifying) a first antibody specific for an antigen of interest (e.g., an influenza virus epitope, for example an epitope of the stalk region of hemagglutinin), and
obtaining (e.g., producing or identifying) an anti-idiotypic antibody against the first antibody. Non-limiting example of the first antibody includes F10, C179, CR6261, CR9114, FI6 and others, for example, as disclosed in Damian C. Ekiert and Ian A. Wilson, Broadly neutralizing antibodies against influenza virus and prospects for universal therapies, Curr Opin Virol. 2012 April: 2(2): 134-141; Jianhua Sui, et al., Structural and functional bases for broad-spectrum neutralization of avian and human influenza A viruses, Nat Struc Mol Bio (published online 22 Feb. 2009) pages 1-9; and Han Zhang, et al., Universal Influenza Vaccines, a Dream to Be Realized Soon Viruses. Viruses (2014) 6, 1974-1991. However, other antibodies can be generated using techniques known in the art.

Certain embodiments of the disclosure relate to isolated or recombinant proteins (e.g., antibodies) and nucleic acids that encode proteins (e.g., antibodies). As used herein, an isolated molecule is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide, or polypeptide.

Also provided are vectors useful for expression of an antibody (e.g., an anti-idiotypic antibody) of the disclosure. In one embodiment the expression vector is suitable for use in mammalian host cells. Mammalian expression vectors can include non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a poly-adenylation site, splice donor and acceptor sites, and transcriptional termination sequences. Commonly used promoters and enhancers are derived from Polyoma, Adenovirus 2, Simian Virus 40 (SV40), and human cytomegalovirus. DNA sequences derived from the SV40 viral genome, for example, SV40 origin, early and late promoter, enhancer, splice, and polyadenylation sites may be used to provide the other genetic elements required for expression of a heterologous DNA sequence. A nucleic acid molecule of the disclosure can be inserted into an appropriate expression vector using standard methods of molecular biology which need not be described in further detail here. The expression vector can include a promoter or promoter/enhancer element that is positioned upstream of the coding nucleic acid molecule that is inserted into the vector. Expression vectors can optionally include at least one coding region for a selection marker and/or gene amplification element.

For expression of an antibody of the disclosure, a vector or vectors containing nucleic acid sequences encoding one or more polypeptide of the antibody can be introduced into a suitable host cell or population of host cells. However, in some embodiments, mRNAs (e.g., synthetic mRNAs) can be delivered that encode an antibody or fragment thereof of the disclosure. In some embodiments, a synthetic mRNA encoding an anti-idiotypic antibody disclosed herein is introduced into a cell (e.g., in vitro or in vivo) to produce the anti-idiotypic antibody in the cell. In some embodiments, the anti-idiotypic antibody is secreted from the cell. Thus, in some embodiments, vaccine compositions are provided herein that include anti-idiotypic antibodies or expression vectors or synthetic mRNAs encoding such antibodies.

The vector or vectors can be introduced into a host cell or cells using any suitable method, including, for example, electroporation, biolistic delivery (e.g., using a gene gun), lipofection, calcium phosphate precipitation, microinjection, viral transduction, nucleofection, sonoporation, magnetofection, and heat shock. Such methods are well known by persons skilled in the art and need not be described here. Following introduction of the vector or vectors into the host cell or cells, the cell or cells are maintained under physiologically suitable conditions suitable for in vitro cell culture, for a period of time sufficient to permit the cell or cells to express the antibody.

As used herein, a host cell is a eukaryotic cell or other cell (e.g., insect cell, prokaryotic cell) suitable for expression of a protein of interest or harboring of a nucleic acid of interest. In some embodiments, the host cell is a mammalian cell. In certain embodiments, the host cell is a mammalian cell line. In some embodiments, the mammalian cell line is non-Ig-secreting myeloma such as NS/0 or Sp2/0-Ag14. In some embodiments, the mammalian cell line is HEK293. In certain embodiments, the mammalian cell line is a Chinese hamster ovary (CHO) line. These and other suitable host cells are available from American Type Culture Collection (ATCC) (Manassas, Va.).

In some embodiments, an antibody is secreted into the culture medium by the cells containing the expression vector or vectors. Secreted expressed antibody can be readily isolated from culture by centrifugation (to remove cells) followed by immunoaffinity separation, for example using protein A or protein G chromatography, and/or using specific antigens to which the antibody binds.

In some embodiments, an anti-idiotypic antibody that mimics or resembles a hemagglutinin antigen is referred to herein as 7G7, c7G7 or m7G7. In some embodiments, an anti-idiotypic antibody that mimics or resembles a hemagglutinin antigen has a heavy chain variable region having an amino acid sequence set forth as:

>7G7 VH protein sequence
(with a leader sequence in brackets)
(SEQ ID NO: 1)
[MEWSGVFIFLLSVTAGVHS]QVQLQQSGVELVRPGTSVKMSCKASGY
TFTNYWIGWAKQRPGHGLEWIGDIYPGGDYTNYNEKFRGKATLTADKS
SSTAYMQFSSLTSEDSAIYYCASLYDGGFAYWGQGTLVTVS

In some embodiments, an anti-idiotypic antibody that mimics or resembles a hemagglutinin antigen has a light chain variable region having an amino acid sequence set forth as:

>7G7 VL protein sequence
(with a leader sequence in brackets)
(SEQ ID NO: 2)
[MDFQVQIFSFLLISASVIMSRG]QIVLTQSPAIMSASPGEKVTISCS
ASSSVSYMYWYQQKPGSSPKPWIYRTSNLASGVPARFSGSGSGTSYSL
TISSMEAEDAATYYCQQFHGFPLTFGAGTKLELK

The foregoing variable sequences comprise mouse leaders. In some embodiments, the anti-idiotypic antibody is engineered as a recombinant chimeric antibody. In such embodiments, the leader sequences may be replaced with different leader sequences (e.g., of the same or different species, e.g., mouse or human). In certain embodiments, leader sequences may be replaced with a standard mouse VL leader (e.g., from an expression vector). In some embodiments, the same leader sequence may be used for both the L and H chains. In some embodiments, different leader sequences may be used for both the L and H chains. In certain embodiments, leader sequences are contained in the mature assembled antibody.

In some embodiments, fragment(s) of a variable region may be used to engineer anti-idiotypic antibodies that substantially retain anti-idiotypic function. In some embodiments, it may be beneficial to engineer such fragments into a new framework to improve one or more therapeutic properties, including to increase the likelihood of anti-anti-idiotypic antibodies being directed against the portion of the anti-idiotypic antibody that mimics the original antigen, rather than a region of the antibody that does not mimic the original antigen.

In some embodiments, one or more CDRs can be engineered into a recombinant or chimeric framework (e.g., a human framework) while retaining the original anti-idiotypic function. For example, in some embodiments, an anti-idiotypic antibody that mimics or resembles a hemagglutinin antigen has one or more heavy chain complementarity determining regions (CDRs) selected from:

VH-CDR1
(SEQ ID NO: 3)
SASSSVSYMY
VH-CDR2
(SEQ ID NO: 4)
RTSNLAS
VH-CDR3
(SEQ ID NO: 5)
QQFHGFPLT

In some embodiments, an anti-idiotypic antibody that mimics or resembles a hemagglutinin antigen has one or more light chain complementarity determining regions (CDRs) selected from:

VL-CDR1
(SEQ ID NO: 6)
GYTFTNYWIG
VL-CDR2
(SEQ ID NO: 7)
DIYPGGDYTNYNEKFRG
VL-CDR3
(SEQ ID NO: 8)
LYDGGFAY

In some embodiments, an anti-idiotypic antibody may be raised against an scFv version of an anti-influenza antibody. In some embodiments, an anti-idiotypic antibody may be raised against an scFv version of an anti-HA antibody. In some embodiments, an anti-idiotypic antibody may be raised against an scFv version of an anti-HA antibody that is fused to mouse Fc (for immunization purposes). In some embodiments, using a Fc of a particular species of animal (e.g., mouse, rabbit) allows the immune response in an animal of that species to be focused on the idiotope. For example, in the context of a mouse antibody, use of a scFV-Fc fusion also allows for hybridoma Mab screening by detection with anti-mouse kappa chain which would not work if it was in a whole antibody format.

In some embodiments, an anti-idiotypic antibody may be raised against an scFv version of an anti-HA antibody having an amino acid sequence as follows:

>amino acid sequence of scFv3
(SEQ ID NO: 9)
QVQLVQSGAEVKKPGSSVKVSCKSSGGTSNNYAISWVRQAPGQGLDWMG
GISPIFGSTAYAQKFQGRVTISADIFSNTAYMELNSLTSEDTAVYFCAR
HGNYYYYSGMDVWGQGTTVTVSSGGGGSGGGGSGGGGISYVLTQPPAVS
GTPGQRVTISCSGSDSNIGRRSVNWYQQFPGTAPKLLIYSNDQRPSVVP
DRFSGSKSGTSASLAISGLQSEDEAEYYCAAWDDSLKGAVFGGGTQLTV

In some embodiments, an anti-idiotypic antibody may be raised against an scFv-Fc version of an anti-HA antibody having an amino acid sequence as follows:

>amino acid sequence of scFv3-mFc (gamma2a)
(SEQ ID NO: 10)
QVQLVQSGAEVKKPGSSVKVSCKSSGGTSNNYAISWVRQAPGQGLDWMG
GISPIFGSTAYAQKFQGRVTISADIFSNTAYMELNSLTSEDTAVYFCAR
HGNYYYYSGMDVWGQGTTVTVSSGGGGSGGGGSGGGGISYVLTQPPAVS
GTPGQRVTISCSGSDSNIGRRSVNWYQQFPGTAPKLLIYSNDQRPSVVP
DRFSGSKSGTSASLAISGLQSEDEAEYYCAAWDDSLKGAVFGGGTQLTV
EPRGPTIKPCPPCKCPAPNLLGGPSVFIFPPKIKDVLMISLSPIVTCVV
VDVSEDDPDVQISWFVNNVEVHTAQTQTHREDYNSTLRVVSALPIQHQD
WMSGKEFKCKVNNKDLPAPIERTISKPKGSVRVPQVYVLPPPEEEMTKK
QVTLTCMVTDFMPEDIYVEWTNNGKTELNYKNTEPVLDSDGSYFMYSKL
RVEKKNWVERNSYSCSVVHEGLHNHHTTKSFSRTPGK;
residues 1-122 are the VH region,
residues 123-139 are the flexible linker, and
residues 140-to the end are the H-CH2 and
CH3 domains of the mouse IgG2a H chain

### Therapeutic Applications

In some embodiments, an anti-idiotypic antibody that mimics an epitope of an influenza virus (e.g., of a capsid protein epitope, or a hemagglutinin epitope, for example an epitope of a fusion region of hemagglutinin) is delivered to a subject (e.g., a human subject) to stimulate an immune response in the subject thereby providing an immunoprotective effect against infection by the influenza virus.

As disclosed herein, anti-idiotypic regions can be provided in any of a number of different configurations. In some embodiments, an anti-idiotypic region is provided in a complete antibody. In some embodiments, an anti-idiotypic region is provided in as a fragment of an antibody, such as a scFv fragment or a single chain antibody or another example disclosed herein. In some embodiments, a fragment of a variable region of an anti-idiotypic antibody is sufficient to produce an immune response against an antigen. For example, in some embodiments, one or more of the following fragments alone or in combination may mimic an antigen or portion thereof: VH-CDR1, VH-CDR2, VH-CDR3, VL-CDR1, VL-CDR2, and VL-CDR3. In some embodiments, an anti-idiotypic region is provided as a fusion with one or more other effector domains to enhance an immunoprotective effect. In some embodiments, an anti-idiotypic region is provided in as a fusion with one or more cytokines.

In some embodiments, a nucleic acid (e.g., a DNA or RNA plasmid or vector, an mRNA, or other nucleic acid) encoding an anti-idiotypic antibody is delivered to a subject (e.g., a human subject) to stimulate an immune response in the subject thereby providing an immunoprotective effect against infection by the influenza virus.

In some embodiments, a combination of an anti-idiotypic antibody or a fragment thereof and a nucleic acid encoding an anti-idiotypic antibody or a fragment thereof are delivered to a subject (e.g., simultaneously, concurrently, or contemporaneously in separate formulations or formulated together). In some embodiments, a composition is provided that comprises two or more anti-idiotypic antibodies, each of which comprises an idiotope mimicking a different influenza virus antigen, or two or more nucleic acids (e.g., expression vector or mRNA) encoding the same. In some embodiments, the different influenza virus antigens are different regions of hemagglutinin or neuraminidase.

Accordingly, in some embodiments, a single anti-idiotypic antibody or fragment thereof is formulated as a vaccine for delivery to a subject (e.g., a human subject). In some embodiments, a nucleic acid encoding a single anti-idiotypic antibody or fragment thereof is formulated as a vaccine for delivery to a subject (e.g., a subject at risk of influenza infection). In some embodiments, a single anti-idiotypic antibody or fragment thereof is formulated together with a nucleic acid encoding a single anti-idiotypic antibody or fragment thereof. In some embodiments, the anti-idiotypic antibody or fragment thereof in the formulation is the same as the anti-idiotypic antibody or fragment thereof that is encoded by the nucleic acid in the formulation. In some embodiments, they are different.

In some embodiments, a combination of two or more different anti-idiotypic antibodies and/or a combination of two or more different nucleic acids encoding different anti-idiotypic antibodies or fragments thereof are formulated as a vaccine for delivery to a subject (e.g., a human subject). In some embodiments, a combination of two or more different anti-idiotypic antibodies (or nucleic acids encoding the same) are provided that mimic different strains of virus. Non-limiting examples of such different strains of virus are provided herein. In some embodiments, a combination of two or more different anti-idiotypic antibodies (or nucleic acids encoding the same) are provided that mimic different epitopes of the same virus (e.g., different portions of a hemagglutinin, e.g., different portions of a stalk region of a hemagglutinin). In some embodiments, combinations of two or more different anti-idiotypic antibodies (or nucleic acids encoding the same) are delivered consecutively (e.g., within 1 hr, 1 day, 1 week, 1 month, 2 months apart). However, in some embodiments, combinations of two or more different anti-idiotypic antibodies (or nucleic acids encoding the same) are delivered simultaneously (e.g., in the same formulation). In some embodiments, combinations of two or more different anti-idiotypic antibodies (or nucleic acids encoding the same) are delivered essentially at the same time but in different formulations. In some embodiments, combinations of two or more different anti-idiotypic antibodies (or nucleic acids encoding the same) are delivered essentially at the same time in different formulations and at different sites or via different routes of administration (e.g., intranasally, intradermally, etc.). In some embodiments, combinations of two or more different anti-idiotypic antibodies (or nucleic acids encoding the same) are delivered essentially at the same time in different formulations and at essentially the same sites or via the same routes of administration (e.g., intranasally, intradermally, etc.).

In some embodiments, one or more different anti-idiotypic antibodies and/or one or more different nucleic acids encoding different anti-idiotypic antibodies or fragments thereof are formulated and/or administered to a subject along with (e.g., simultaneously, concurrently, or contemporaneously) one or more adjuvants.

### Pharmaceutical Compositions and Administration

Subjects according to methods disclosed herein include any subject with an appropriate immune system including mammalian and avian subjects. Non-limiting examples of subjects include humans, non-human primates, rodents (e.g., rats, mice), agricultural mammals (e.g., pigs, horses, cows), agricultural birds (e.g., chickens, hens), and pets (e.g., dogs, cats).

In some embodiments, a subject is a human. In some embodiments, a subject is a human at risk for a flu infection. In some embodiments, a subject at risk for a flu infection is a young human (a juvenile). In some embodiments, a subject at risk for a flu infection is an elderly human. In some embodiments, a subject at risk for a flu infection is under 12 years of age. In some embodiments, a subject at risk for a flu infection is under 18 years of age. In some embodiments, a subject at risk for a flu infection is in the range of 18 to 65 years of age. In some embodiments, a subject at risk for a flu infection is older than 65 years of age. In some embodiments, a subject at risk for a flu infection is older than 80 years of age. In some embodiments, a subject at risk for a flu infection is an immunocompromised human (e.g., due to a disease or a side effect of a therapeutic treatment).

As used herein, the term “treat” or “treating” refers to preventing, slowing or halting the progression of, or to reducing or eliminating, a disease or one or more symptoms of a disease (e.g., influenza) in a subject. It should be appreciated that subjects can be immunized whether or not they have symptoms or are suspected of having a flu infection.

In some embodiments, the anti-idiotypic antibody (including antigen binding fragments) and/or nucleic acid of the disclosure that is being administered is adapted for the recipient subject (e.g., humanized for a human subject, etc.).

In some embodiments, the anti-idiotypic antibody and/or nucleic acid is provided along with suitable adjuvants, excipients, or carriers. In some embodiments, the vaccine preparation is sterilized (e.g., using any suitable technique). Accordingly, also provided are compositions that include an antibody and/or nucleic acid of the disclosure and suitable carrier. In one embodiment, the composition is a pharmaceutical composition that includes an antibody and/or nucleic acid of the disclosure and a pharmaceutically acceptable carrier. In some embodiments, an adjuvant is an aluminum salt, such as aluminum hydroxide, aluminum phosphate, or aluminum potassium sulfate. In some embodiments, an adjuvant is monophosphoryl lipid A. Other adjuvants may be used such as saponin adjuvants (e.g., saponins from Quillaja, Soybean, or Polygala senega) oil-water emulsion based adjuvants, calcium phosphate hydroxide, squalene, thimerosal, or detergent based adjuvants, such as Quil A.

The term “pharmaceutically-acceptable carrier” means one or more compatible solid or liquid fillers, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with other compounds, and with each other, in a manner such that there is no interaction which would substantially impair the desired pharmaceutical efficiency.

In some embodiments, a preparation or formulation described herein has been sterilized (e.g., by filtration, UV irradiation, or other suitable technique).

In some embodiments, vaccine compositions may be cryopreserved. Accordingly, in some embodiments, vaccine compositions may be formulated with a cryopreservative such as, for example, DMSO, ethylene glycol, glycerol, 2-methyl-2,4-pentanediol (MPD), propylene glycol or sucrose.

As used herein, an “effective amount” refers to the amount necessary or sufficient to realize a desired biologic effect. Combined with the teachings provided herein, by choosing among the various active compounds and weighing factors such as potency, relative bioavailability, patient body weight, severity of adverse side-effects and preferred mode of administration, an effective therapeutic treatment regimen can be planned which does not cause substantial toxicity and yet is effective to treat the particular subject. The effective amount for any particular application can vary depending on such factors as the disease or condition being treated, the particular active agent being administered, the size of the subject, or the severity of the disease or condition. One of ordinary skill in the art can empirically determine the effective amount of a particular active agent and/or other therapeutic agent without necessitating undue experimentation. In some embodiments, a dose may be used that represents the highest safe dose according to some medical judgment. Multiple doses per week, per month, per year or another suitable frequency may be performed to achieve appropriate immune responses. Appropriate systemic levels can be determined by, for example, measurement of the subject's peak or sustained immune reactivity to a particular antigen or anti-idiotypic antibody.

For any antibody or nucleic acid described herein, a therapeutically effective amount can be initially determined from animal models. A therapeutically effective dose can also be determined from human data for antibodies which have been tested in humans and for compounds which are known to exhibit similar pharmacological activities, such as other related active agents. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

For use in therapy, formulations of the disclosure can be administered in pharmaceutically acceptable solutions, which may routinely contain pharmaceutically acceptable concentrations of salt, buffering agents, preservatives, compatible carriers, adjuvants, and optionally other therapeutic ingredients.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

For use in therapy, an effective amount of the antibody can be administered to a subject by any mode that delivers the antibody to the desired target tissue. Administering the pharmaceutical composition of the present disclosure may be accomplished by any means known to the skilled artisan. Routes of administration include but are not limited to oral, intranasal, intramuscular, intravenous and subcutaneous.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For subcutaneous administration, agents can be chosen that do not cause local skin irritation. In some embodiments, agents are generally isotonic and do not contain high levels of harsh detergents.

Alternatively, the active compounds may be in powder form for constitution with a suitable vehicle, e.g., sterile pyrogen-free water, before use.

In some embodiments, a subject is dosed with an anti-idiotypic antibody and/or nucleic acid of the disclosure annually. In some embodiments, a subject is dosed with an anti-idiotypic antibody and/or nucleic acid of the disclosure seasonally (e.g., in late autumn/early winter). In some embodiments, annual dosing is not necessary. In some embodiments, seasonal flu viruses boost a prior anti-anti-idiotypic response each time a patient is exposed.

The present disclosure is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1: Production of Anti-Idiotypic Antibodies Useful as an Influenza Vaccine

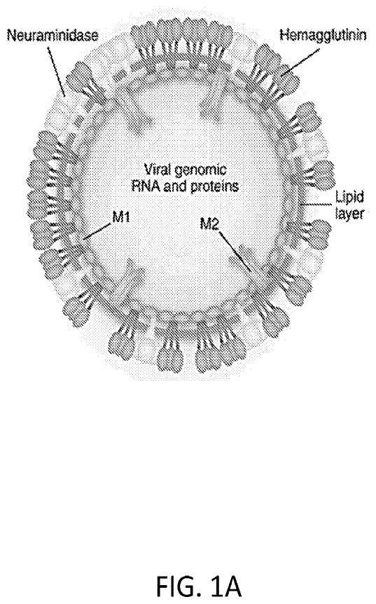

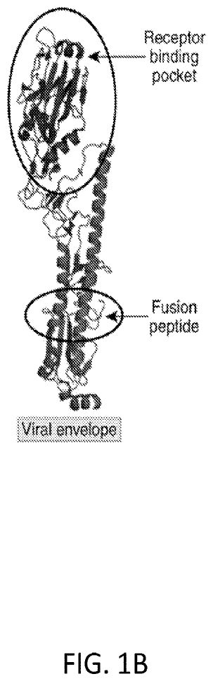

A starting human antibody (F10), modified to contain mouse IgG Fc regions—(F10-mγ2a) was obtained that neutralizes many of the most dangerous Type A influenza (bird flu, swine flu, Spanish flu) by binding to the conserved stalk region (blocks virus fusion—not cell binding). FIG. 1A illustrates an influenza viral particle having surface hemagglutinin (HA). FIG. 1B depicts the receptor binding pocket and fusion peptide portions, which comprises the conserved stalk region, of HA. The general process of the anti-idiotypic antibody production is depicted in FIG. 2.

A second anti-HA stalk region antibody (CR9114) with broader neutralization activity than F10 was converted to an scFv format by the insertion of a (gly4ser)3 sequence between the N terminal VL and C terminal VH and fusing the resulting cDNA to the sequence encoding a mouse IgG2a Fc region. The complete sequence was inserted into an expression vector and used to transiently transfect HEK 293 cells growing in 100 mm plates. After three days the supernatant was passed through a protein A column and the antibody was eluted with low pH buffer and then dialyzed into PBS using an diafiltration centrifuge tube (EMD Millipore). The concentration was determined using an anti-mouse antibody ELISA. The same vector was used to stably transfect CHO cells and individual expressing clones were identified and expanded for frozen cell storage and the generation of additional protein for immunization of mice to generate anti-idiotypic antibodies.

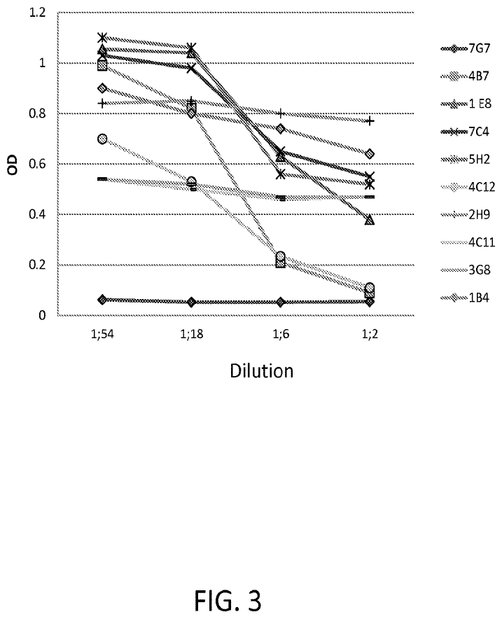

An scFv version of the F10-mγ2a antibody (referred to as PR1) was produced and expressed in NS/0 cells. PR1 was used to immunize mice and generate a hybridoma library (AbPro Labs). Primary screening was performed which involved measuring binding of mouse antibodies in hybridoma supernatants to PR1 on ELISA plate using anti-mouse k chain-HRP for detection. Secondary screening was performed to test blocking of biotinylated scFv-F10-mγ2a (PR1-BIO) to HA coated on ELISA plates, as depicted in FIG. 3. A high affinity binder with strong inhibitory activity was identified. This antibody is identified as 7G7. Mouse antibody (IgG1) produced from hybridoma cells in culture and purified using protein G, was used to immunize mice.

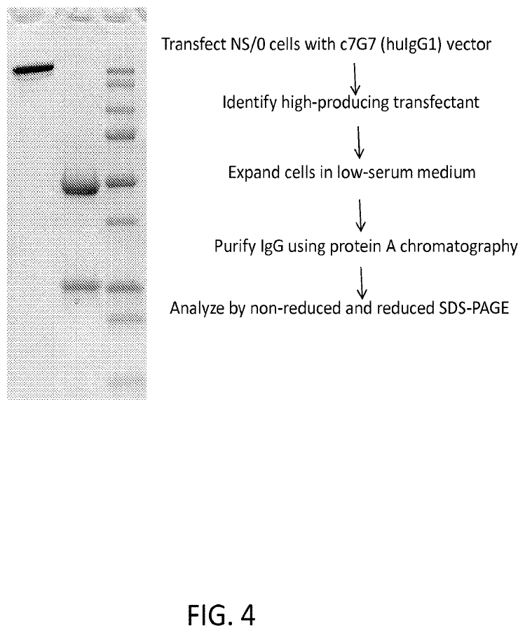

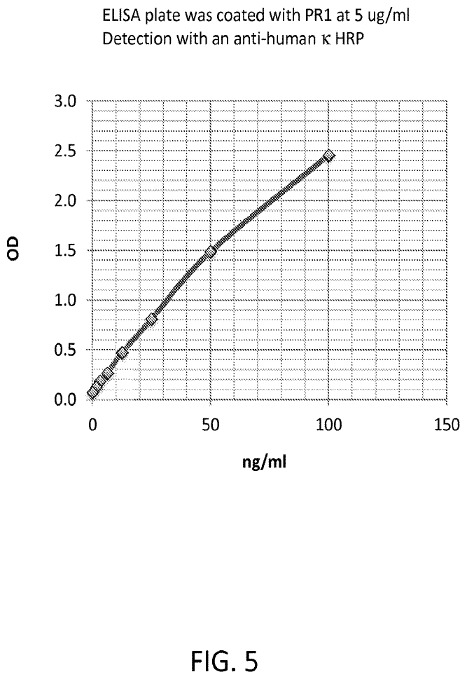

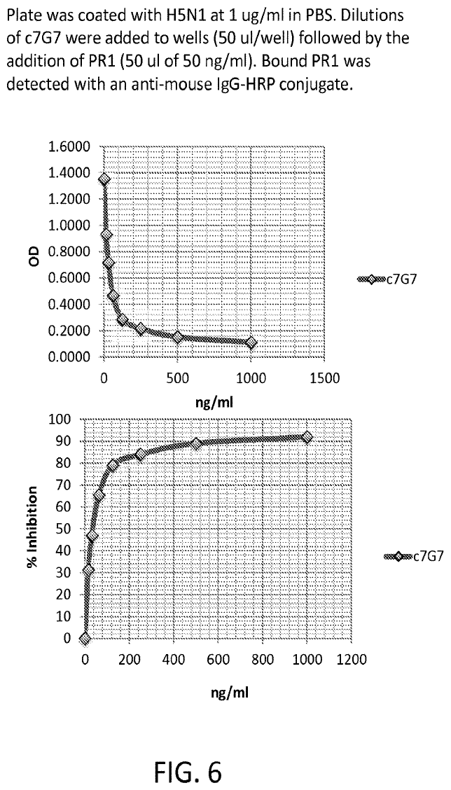

Variable regions of the 7G7 antibody were cloned and sequenced (Blue Sky Biotech), and a chimeric mouse-human antibody expressed and purified (c7G7). FIG. 4 illustrates results of an SDS-PAGE assay showing a purified chimeric anti-idiotypic antibody (referred to as c7G7 under reducing (R) and non-reducing (NR) conditions). FIG. 5 shows results of an ELISA assay showing binding of c7G7 to PR1. FIG. 6 shows results of an ELISA assay showing inhibition of binding HA protein to PR1 by 7G7.

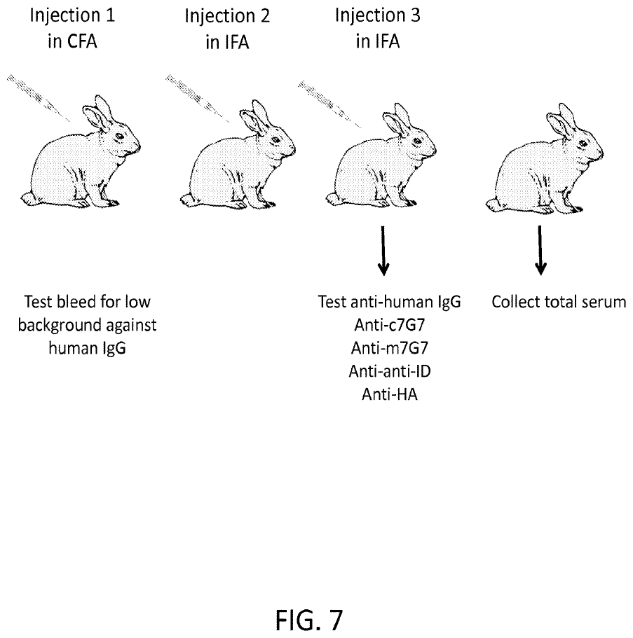

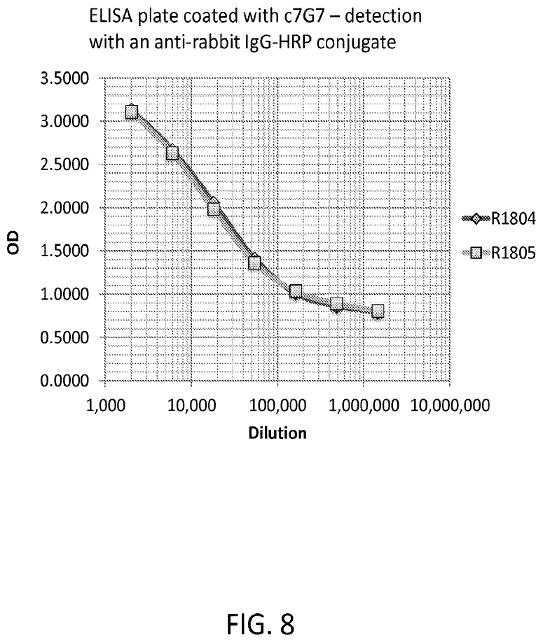

Chimeric 7G7 (c7G7) antibody was used to immunize rabbits (R1804 and R1805). FIG. 7 illustrates the immunization protocol used. FIG. 8 shows the response of c7G7 immune rabbit sera to the c7G7 antibody containing human C regions. Generally, rabbits make strong antibody responses to foreign (e.g. human) IgG antibody C regions so it's important to see if responses are being made to the V regions containing the anti-idiotope as well.

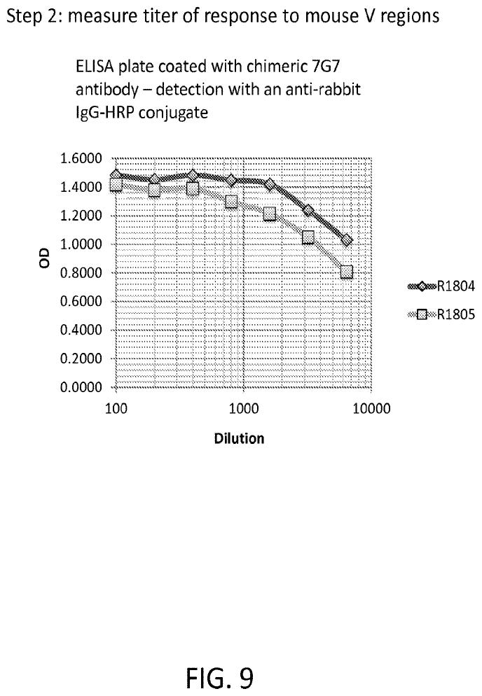

FIG. 9 shows the response of c7G7 immune rabbit sera to the mouse 7G7 antibody, which shares only the mouse V regions with the antigen it was immunized with c7G7. ELISA plates were coated with mouse 7G7 antibody. Sera was obtained from two rabbits (R1804 and R1805) that had been immunized with c7G7. ELISAs were performed with serial dilutions of the sera from the two rabbits and binding to immobilized 7G7 was detected using anti-rabbit IgG-HRP conjugates. Results show dose dependent binding of the sera to the plates indicating that an anti-anti-idiotypic response was produced in both immunized rabbits.

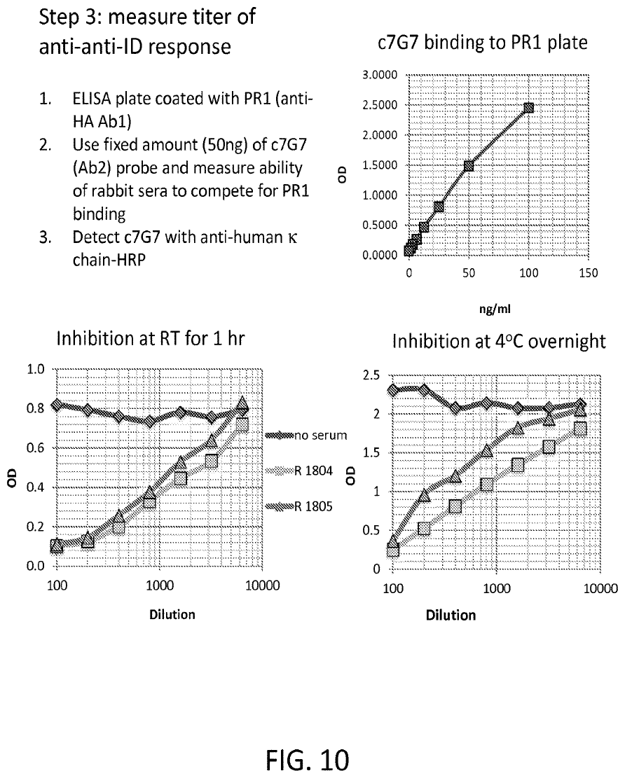

FIG. 10 shows the response of 7G7 immune rabbit sera to 7G7 in the specific region needed to bind to PR1 (the anti-anti-idiotypic response). ELISA plates were coated with PR1 (anti-HA Ab1). Dose dependent binding of c7G7 to the PR1 coated plates was assessed as a quality control step (upper right panel). A fixed amount of (50 ng) of c7G7 (Ab2) probe was used to measure ability of rabbit sera to compete for PR1 binding. c7G7 binding was detected with anti-human k chain-HRP at different dilutions following incubation at RT for 1 hour at room temperature or 4° C. overnight (lower panels). Dose dependent inhibition of binding of c7G7 to PR1 coated plates was observed in both sera (R1804 and R1805).

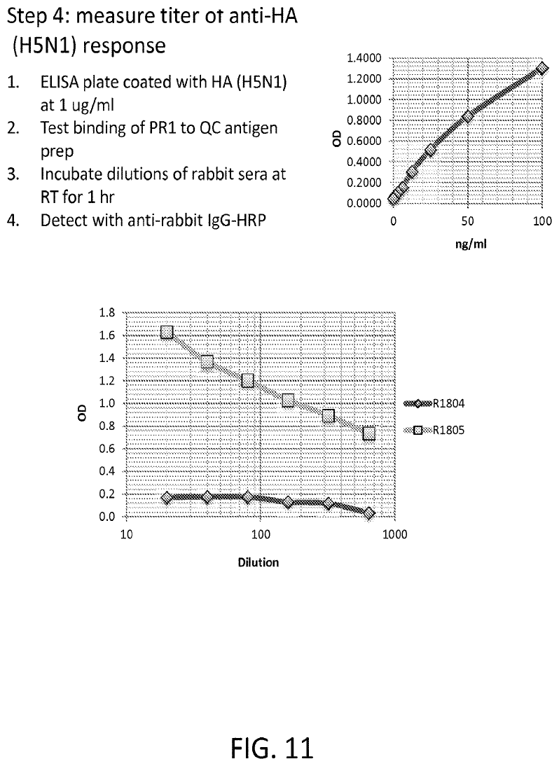

FIG. 11 shows the rabbit IgG response of 7G7 immune rabbit sera to H5N1 HA protein. The rabbit IgM response can also be measured using specific secondary anti-rabbit IgM antibodies. ELISA plates were coated with HA (H5N1) at 1 μg/ml. Dose dependent binding of PR1 to the HA coated plates was detected as quality control step (upper right panel). Dilutions of rabbit sera (R1804 and R1805) were incubated with the ELISA plates at RT for 1 hr and binding was detected with anti-rabbit IgG-HRP. Dose dependent inhibition of binding of PR1 to HA coated plates was observed in both sera (R1804 and R1805).

Injecting a mouse antibody into a mouse helps select for a response against the idiotype (other regions are seen as “self”). 7G7 antibody (mouse IgG1) was grown in low serum medium and purified using protein G. GenScript was contracted to immunize 3 mice with 100 ug/dose. Day 1 dose was formulated in CFA, day 14 and day 35 were in IFA. Sera were collected on day 45 and tested for binding to HA (H5N1). One mouse had high background in pre-immune serum (mouse 2).

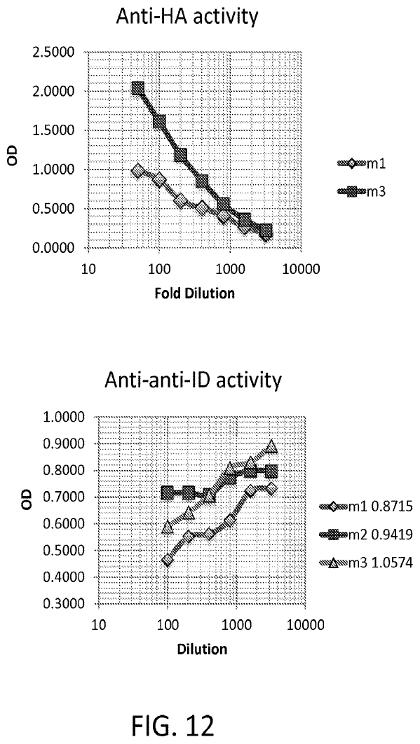

Two mouse sera were positive for binding when titrated on an HA-coated plate, based on detection with anti-mouse IgG-HRP, as illustrated in FIG. 12. Anti-anti-ID activity was measured on the same sera by showing the blocking of binding of 7G7 to plate-bound PR1.

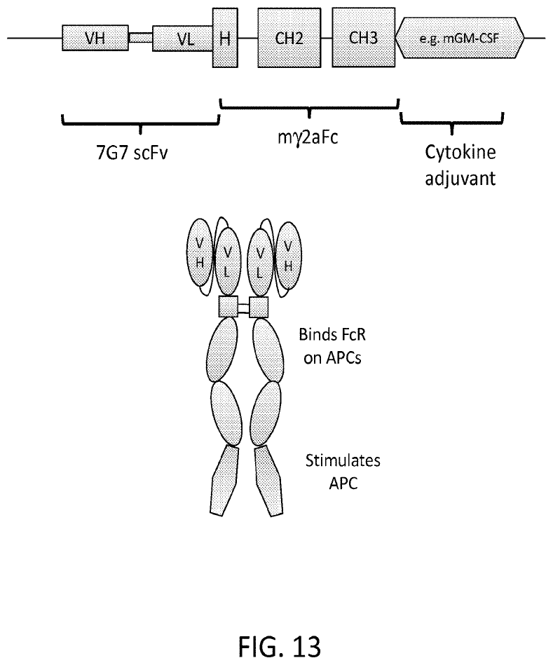

An example of an anti-idiotypic antibody construct was developed to test the potential as a vaccine in mouse models that incorporate the 7G7 scFv region and mγ2aFc region and a cytokine adjuvant, as depicted in FIG. 13.

>Amino acid sequence of 7G7-mFc-mGM-CSF
(SEQ ID NO: 11)
QVQLQQSGVELVRPGTSVKMSCKASGYTFTNYWIGWAKQRPGHGLEWI
GDIYPGGDYTNYNEKFRGKATLTADKSSSTAYMQFSSLTSEDSAIYYC
ASLYDGGFAYWGQGTLVTVSGGGGSGGGGSGGGGIQIVLTQSPAIMSA
SPGEKVTISCSASSSVSYMYWYQQKPGSSPKPWIYRTSNLASGVPARF
SGSGSGTSYSLTISSMEAEDAATYYCQQFHGFPLTFGAGTKLELKEPR
GPTIKPCPPCKCPAPNLLGGPSVFIFPPKIKDVLMISLSPIVTCVVVD
VSEDDPDVQISWFVNNVEVHTAQTQTHREDYNSTLRVVSALPIQHQDW
MSGKEFKCKVNNKDLPAPIERTISKPKGSVRVPQVYVLPPPEEEMTKK
QVTLTCMVTDFMPEDIYVEWTNNGKTELNYKNTEPVLDSDGSYFMYSK
LRVEKKNWVERNSYSCSVVHEGLHNHHTTKSFSRTPGAPTRSPITVTR
PWKHVEAIKEALNLLDDMPVTLNEEVEVVSNEFSFKKLTCVQTRLKIF
EQGLRGNFTKLKGALNMTASYYQTYCPPTPETDCETQVTTYADFIDSL
KTFLTDIPFECKKPVQK

In some embodiments, this construct has the potential advantage of surviving in vivo for a longer time, as well as binding to Fc receptors on antigen-presenting cells (APC) and utilizing GM-CSF to increase the maturation of the most potent APCs—dendritic cells. Alternatively, the scFv region alone, in combination with an adjuvant, could be used as a vaccine. Likewise, the c7G7 chimeric antibody could be used in humans, together with adjuvant, or fused to an immunostimulatory cytokine. Nucleic acids encoding the 7G7 V regions as an scFv or other antibody format (e.g. DNA vectors expressing the protein, or synthetic messenger RNA (e.g., a synthetic messenger RNA containing at least one non-natural nucleotide and/or internucleotide linkage) could be used as a vaccine, either alone or as part of a prime-boost strategy. Due to the broad cross-reactivity of this approach, priming of any of the above vaccine formats, followed by boosting with a more traditional monovalent or multivalent influenza vaccine (e.g. killed virus vaccine) could be highly effective at inducing a potent and broad based immune response to the stalk region fusion domain. Such an effect was observed using a DNA vaccine encoding H5N1 HA as the priming dose, followed by inactivated H5N1 virus, in which case there were increased responses to the stalk region, compared to virus immunization alone (Julie E. Ledgerwood, et al. DNA priming and influenza vaccine immunogenicity: two phase 1 open label randomized clinical trials. Lancet Infect Dis 2011; 11: 916-24).

ELISA of Rabbit Antisera and scFv-Fc s Using HA-Transfected M21 Cells

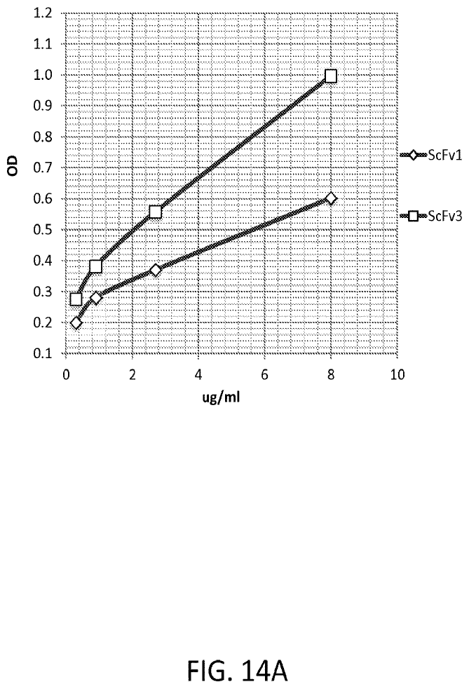

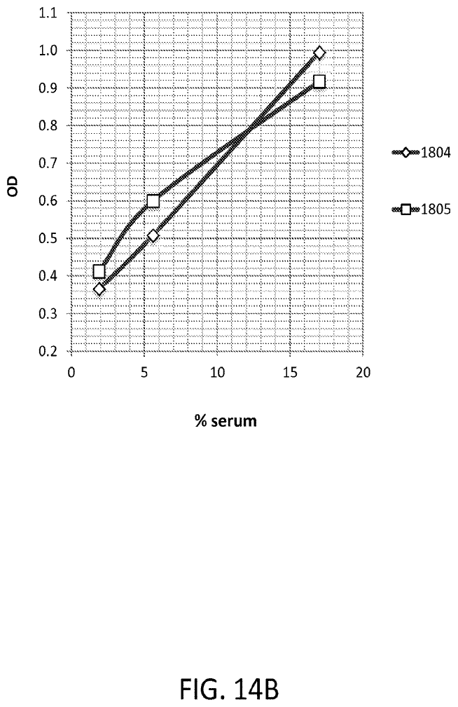

M21 cells stick very well to 96-well plates and monolayers stay intact after several washes and formaldehyde fixation making them useful for cell based ELISAs. A monolayer of M21 cells growing in RPMI medium containing 10% FBS, 2 mM glutamine and 1% penicillin-streptomycin (growth medium) in a 100 mm plate was trypsinized and then mixed with medium to stop proteolysis. Cells were counted and diluted to 2×10⁵ cells/ml and 0.2 ml was added to 4 rows of a 96-well plate (32 wells in all). The cells were about 80-90% confluent 24 hrs. after seeding. They were transfected by making the following transfection mixture and adding 10 μl of the final solution to each well using a micropipettor. A 350 μl mixture consisted of 175 μl DNA solution containing 4 μg HA vector DNA (Sino Biologicals) and 163 μl Opti-MEM, mixing and then adding 8 μl P3000. The second solution contained 170 μl Opti-MEM and 5 μl LF3000 that was mixed and allowed to sit for 5 minutes before use. The two solutions were mixed, pipetted gently and incubated for 5 minutes before adding 10 μl/well to cells in 0.18 ml volume. The plate was incubated for 24 hr before using for the ELISA. The culture medium was removed by flicking the contents and 100 μl PBS/well was added and removed, followed by 100 μl/well of 4% formaldehyde in PBS. After 15 minutes, the plate was washed twice with PBS. Antibody solutions or antiserum were diluted with PBS-1% FBS to 5 μg/ml or 50%, respectively. 150 μl aliquots were added to the top wells and diluted 3-fold by transfer of 50 μl through 100 μl of PBS-1% FBS. Four concentrations were tested for each test article. The plate was incubated at RT for 1 hr, after which the wells were washed with PBS and then 100 μl of a mixture of goat anti-mouse-HRP and goat anti-rabbit HRP (1:2000 each) was added to each well for 1 hr. After washing three times, TMB solution was added, followed by 0.1 M HCl as the stop solution. Absorbance was measured at 450 nm using a GENios Pro plate reader as an indicator of antibody binding. Results, as depicted in FIG. 14A and FIG. 14B, show that both human antibodies formatted as scFv fusions to mouse IgG2a Fc bind H5N1 Vietnam HA transiently transfected into M21 melanoma cells with the scFv3 (antibody CR9114) showing stronger binding than scFv1 (antibody F10). The rabbit antisera from animals immunized with anti-idiotypic antibody c7G7 both showed similar binding to M21 cells expressing HA with the highest binding at the least diluted concentration (around 16.7% serum). When a higher concentration of 50% was tested, binding decreased due to serum component interference (not shown).

### Syncytia Inhibition Assay

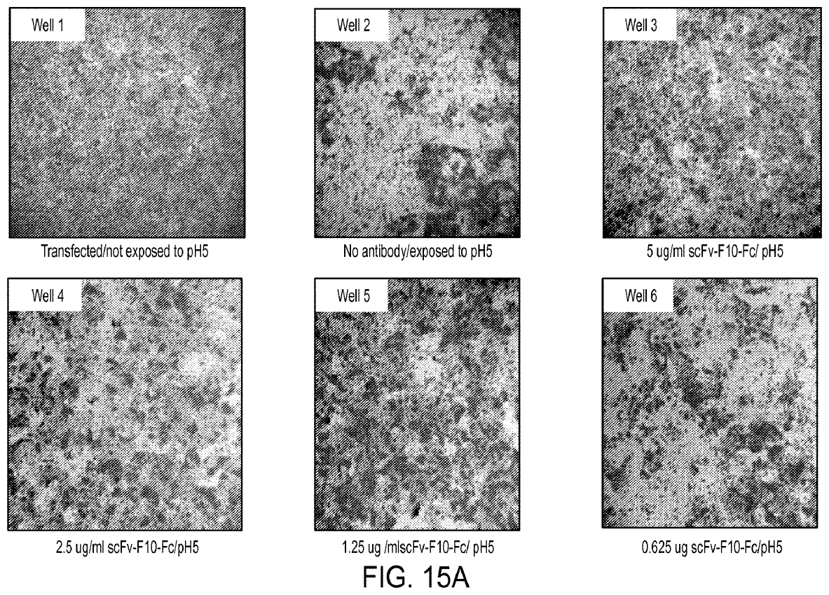

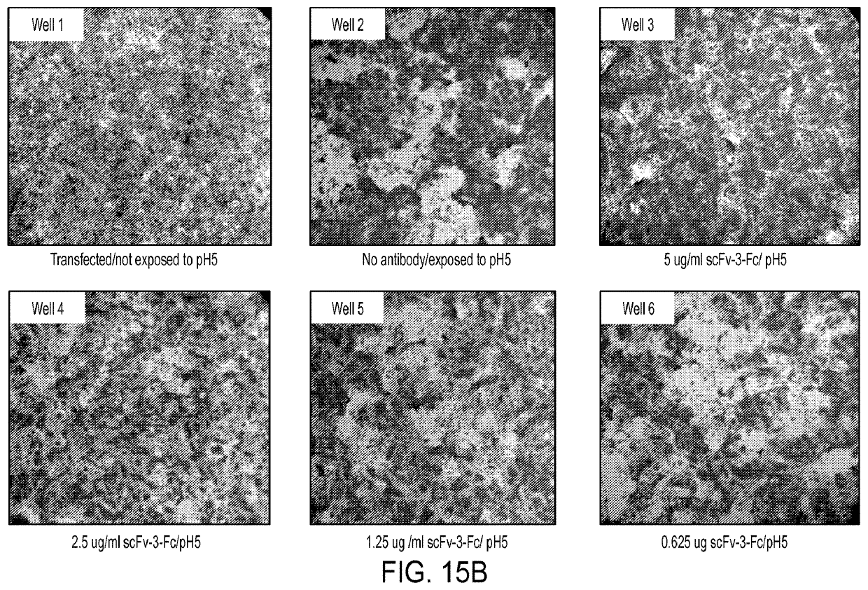

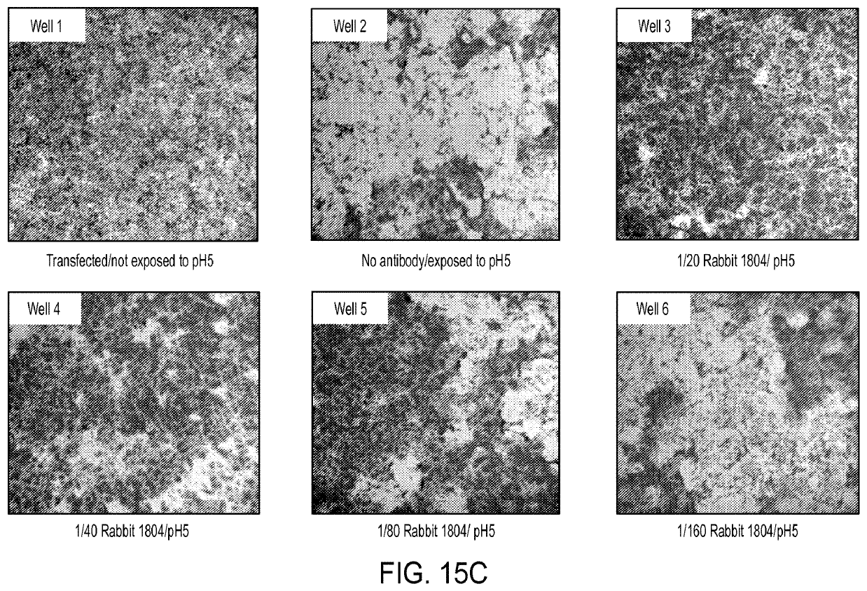

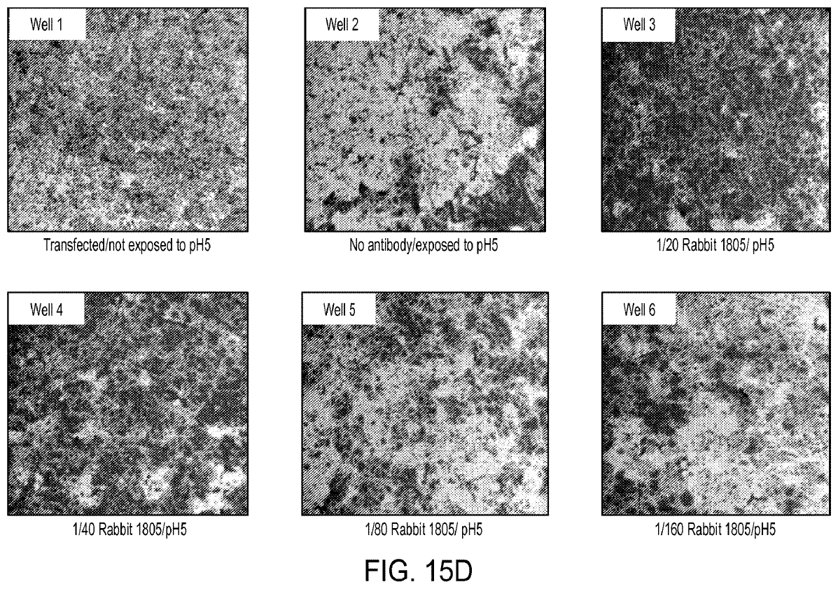

HeLA cells expressing certain HA molecules on their cell surface can be induced to form syncytia by a short exposure to a low pH buffer. Antibodies interacting with the conserved stalk region that neutralize infectivity by preventing envelope fusion (rather than cell binding) can also block syncytia formation. Therefore, this cell culture assay can be used to screen anti-stalk region antibodies for neutralizing activity, as well as the serum of animals vaccinated with the intention of inducing this class of antibody. HeLa cells in DMEM containing 10% FBS, 2 mM glutamine and 1% penicillin-streptomycin (growth medium) were trypsinized and seeded in wells of a 24 well plate at 1×10⁵ cells/ml in 0.5 ml per well. The next day (24 hr) they were transfected with an expression vector obtained from Sino Biologicals that has been codon optimized for expression of the HA H5N1 Vietnam isolate (catalog number VG11062-UT). The transfection mixture contained 3 μg of HA vector DNA and 6 ul of P3000 enhancer in 0.6 ml of Opti-MEM that was mixed with an equal volume of lipofectamine 3000 in 0.6 ml Opti-MEM (all from Thermo Fisher Scientific). After 5 minutes, 50 μl of the mixture was added to each well of the 24-well plate and the cells were incubated for 48 hr. The first two rows (top to bottom) of wells of the plate had no additions at this time but the next 4 rows had 2-fold titrations of either purified anti-HA stalk antibody of rabbit immune serum (immunized with c7G7 antibody). For the antibodies, a stock solution of 50 μg/ml was used while a Y2 dilution of rabbit antisera was use for the highest concentration. Each well received 50 μl of each dilution resulting in final concentrations of 5, 2.5, 1.25 and 0.625 mg/ml of the test antibodies or 50 μl of each serum dilution resulting in final dilutions of 1/20, 1/40, 1/80 and 1/160. After a 90 minute incubation, the content of the wells of wells 2-6 were removed by aspiration and 0.5 ml fusion buffer (150 mM NaCl, 10 mM HEPES, pH5.0) was added. After 5 minutes, the contents of all wells were removed and replaced with fresh growth medium. Approximately 4 hours later extensive syncytia had formed in the positive control cells (well 2) while the negative control cells (well 1) appeared normal, as shown in FIGS. 15A-15D. The cells shown in these images were fixed for 15 minutes with 4% paraformaldehyde, rinsed twice with PBS and then stained for 15 minutes with 1% crystal violet, after which they were washed several times with water and air dried. As shown in FIGS. 15A-15B, the wells that had received the highest concentration (5 mg/ml) of either the scFv-F10-mFc or the scFv-3-mFc were significantly protected from syncytia-induced cytotoxicity and this protection decreased upon dilution. A dose dependent decrease in protection was observed (wells 3-6). The immune serum from the two rabbits immunized with c7G7 anti-idiotypic antibody showed nearly the same level of protection at the 1/20 dilution as the 5 μg/ml concentration of these antibodies, as shown in FIGS. 15C-15D. This shows that the induced rabbit antibodies that have been shown to bind HA H5 in transfected M21 cells are directed to the original stalk region epitopes of the F10 antibody.

The sera of immunized animals or patients is assessed for influenza neutralizing antibodies using well established methods such as the microneutralization (MN) assay that has been established by the World Heath Organization (WHO). Briefly, 100 TCID50 (median tissue culture infectious doses) of virus in equal volume is mixed with two-fold serial dilutions of antisera (heat-inactivated at 56° C.) in 96-well plates and incubated for 1 h at 37° C. Indicator MDCK cells (1.5×10⁴ cells per well) are added to the plates, followed by incubation at 37° C. for 20 h. To establish the endpoint, the cell monolayers are washes with PBS, fixed in acetone and the viral antigen detected by indirect ELISA with a mAb against influenza A NP (A-3, Accurate). In some embodiments, the result of immunization is that increased dilutions of the sera are needed to reach the endpoint of titration at which the antibodies do not inhibit virus infection and replication, relative to the pre-dose serum control. This amount of dilution should roughly parallel the titration of HA binding activity and/or anti-anti-idiotypic responses measured in those assays. The MN assay can be used to test whole antisera or antibodies purified by, for example, protein A column chromatography (for certain IgG isotypes). Alternatively, anti-anti-idiotypic antibodies in the sera can be captured by binding to a column to which has been coupled the anti-idiotypic antibody (e.g. 7G7).

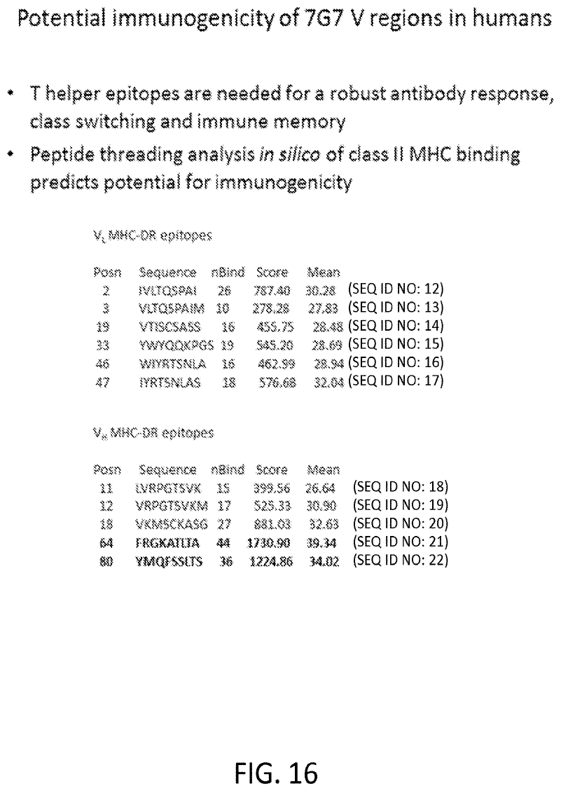

In order to optimize the anti-idiotypic vaccine it's important to determine whether the protein sequence is going to be immunogenic in humans, and if not, to design ways to make it so (e.g. conjugating it to an immunogenic protein such as keyhole limpet hemocyanin [KLH]). DNA sequences of the variable regions of the 7G7 antibody were determined and used to predict the protein sequences. Peptide threading analysis was performed using in silico methods to predict class II MHC binding sites, as depicted in FIG. 16. Methods for MHC binding site prediction are described, for example, in De Groot A S, Knopp P M, Martin W. De-immunization of therapeutic proteins by T-cell epitope modification. Dev Biol (Basel). 2005; 122:171-94. Other appropriate methods for MHC binding site prediction may be used.

Potential peptide degradation products were tested in silico for the strength of binding to each of 50 HLA-DR molecules and the number of binding molecules (nBind) and the average binding strength were calculated. FIG. 16 illustrates the potential immunogenicity of 7G7 V regions in humans based on this analysis. Two particularly strong epitopes in the VH region are bolded. These each bind to the majority of HLA-DR molecules in the human population with high affinity, therefore providing a strong helper T cell signal to promote a B cell growth, differentiation and antibody secretion.

### EQUIVALENTS

The foregoing written specification is considered to be sufficient to enable one skilled in the art to practice the disclosure. The present disclosure is not to be limited in scope by examples provided, since the examples are intended as a single illustration of one aspect of the disclosure and other functionally equivalent embodiments are within the scope of the disclosure. Various modifications of the disclosure in addition to those shown and described herein will become apparent to those skilled in the art from the foregoing description and fall within the scope of the appended claims. The advantages and objects of the disclosure are not necessarily encompassed by each embodiment of the disclosure.

1. An anti-idiotypic antibody comprising an idiotope mimicking an influenza virus antigen.

2. The anti-idiotypic antibody of claim 1, wherein the anti-idiotypic antibody binds specifically to an idiotope of an anti-influenza antibody selected from: F10, C179, CR6261, CR9114, and F16 antibodies.

3. The anti-idiotypic antibody of claim 1, wherein the influenza virus antigen comprises at least a portion of hemagglutinin.

4. The anti-idiotypic antibody of claim 1, wherein the influenza virus antigen comprises a three-dimensional immunogenic region of hemagglutinin.

5. The anti-idiotypic antibody of claim 1, wherein the influenza virus antigen is within the stalk region of hemagglutinin.

6. The anti-idiotypic antibody of claim 1, wherein the anti-idiotypic antibody is effective for inducing an immune response that comprises production of antibodies that specifically bind to hemagglutinin and block cell entry by the virus in a subject.

7. The anti-idiotypic antibody of claim 3, wherein hemagglutinin is of a subtype selected from subtypes H1-16.

8. The anti-idiotypic antibody of claim 1, wherein the influenza virus is an influenza A viruses.

9. The anti-idiotypic antibody of claim 1, wherein the influenza virus is selected from the group consisting of H5N1, H1N1, H2N2, H6N1, H6N2, H8N4, H9N2 and H3N2 influenza viruses.

10. The anti-idiotypic antibody of claim 1, wherein the antibody is a monoclonal antibody.

11. The anti-idiotypic antibody of claim 1, wherein the antibody comprises a Fab, Fab′, F(ab′)2, scFv, Fv, dsFv diabody, or Fd fragment.

12. The anti-idiotypic antibody of claim 1, coupled to a cytokine adjuvant.

13. The anti-idiotypic antibody of claim 12, wherein the cytokine adjuvant is GM-CSF.

14. The anti-idiotypic antibody of claim 1, comprising a V_{L} MHC-DR epitope selected from the group consisting of: IVLTQSPAI (SEQ ID NO: 12), VLTQSPAIM (SEQ ID NO: 13), VTISCSASS (SEQ ID NO: 14), YWYQQKPGS (SEQ ID NO: 15), WIYRTSNLA (SEQ ID NO: 16) and IYRTSNLAS (SEQ ID NO: 17).

15. The anti-idiotypic antibody of claim 1, comprising a V_{H} MHC-DR epitope selected from the group consisting of: LVRPGTSVK (SEQ ID NO: 18), VKMSCKASG (SEQ ID NO: 19), VRPGTSVKM (SEQ ID NO: 20), FRGKATLTA (SEQ ID NO: 21) and YMQFSSLTS (SEQ ID NO: 22).

16. A vaccine composition comprising the anti-idiotypic antibody of any preceding claim and a pharmaceutically acceptable carrier.

17. The vaccine composition of claim 16, further comprising an adjuvant.

18. A method of inducing an immune response in subject, the method comprising administering to the subject the vaccine composition of claim 16.

19. The method of claim 18, wherein the immune response is a protective immune response.

20-41. (canceled)

42. A method of producing a vaccine for influenza virus comprising:
providing a first antibody specific for an influenza virus antigen within a stalk region of hemagglutinin; and
producing a second antibody against the first antibody;
detecting binding of the first antibody to the influenza virus antigen within the stalk region of hemagglutinin in the presence and absence of the second antibody;
immunizing an animal with the second antibody if the binding of the first antibody to the influenza virus antigen within the stalk region of hemagglutinin is decreased in the presence of the second antibody relative to in the absence of the second antibody;
detecting binding of a serum antibody from the immunized animal to a influenza virus hemagglutinin; and
selecting the second antibody as a component of the vaccine if the second antibody is an anti-idiotypic antibody comprising an idiotope mimicking the influenza virus antigen within the stalk region of hemagglutinin,
wherein the second antibody is an anti-idiotypic antibody comprising an idiotope mimicking the influenza virus antigen within the stalk region of hemagglutinin if the serum antibody binds to the influenza virus hemagglutinin.


SEQUENCE LISTING (25 sequences)

SEQ ID NO: 1 - protein, 135 aa, Artificial Sequence
  Synthetic Polynucleotide
MEWSGVFIFLLSVTAGVHSQVQLQQSGVELVRPGTSVKMSCKASGYTFTNYWIGWAKQRPGHGLEWIGDIYPGGDYTNYNEKFRGKATLTADKSSSTAYMQFSSLTSEDSAIYYCASLYDGGFAYWGQGTLVTVS

SEQ ID NO: 2 - protein, 128 aa, Artificial Sequence
  Synthetic Polynucleotide
MDFQVQIFSFLLISASVIMSRGQIVLTQSPAIMSASPGEKVTISCSASSSVSYMYWYQQKPGSSPKPWIYRTSNLASGVPARFSGSGSGTSYSLTISSMEAEDAATYYCQQFHGFPLTFGAGTKLELK

SEQ ID NO: 3 - protein, 10 aa, Artificial Sequence
  Synthetic Polynucleotide
SASSSVSYMY

SEQ ID NO: 4 - protein, 7 aa, Artificial Sequence
  Synthetic Polynucleotide
RTSNLAS

SEQ ID NO: 5 - protein, 9 aa, Artificial Sequence
  Synthetic Polynucleotide
QQFHGFPLT

SEQ ID NO: 6 - protein, 10 aa, Artificial Sequence
  Synthetic Polynucleotide
GYTFTNYWIG

SEQ ID NO: 7 - protein, 17 aa, Artificial Sequence
  Synthetic Polynucleotide
DIYPGGDYTNYNEKFRG

SEQ ID NO: 8 - protein, 8 aa, Artificial Sequence
  Synthetic Polynucleotide
LYDGGFAY

SEQ ID NO: 9 - protein, 245 aa, Artificial Sequence
  Synthetic Polynucleotide
QVQLVQSGAEVKKPGSSVKVSCKSSGGTSNNYAISWVRQAPGQGLDWMGGISPIFGSTAYAQKFQGRVTISADIFSNTAYMELNSLTSEDTAVYFCARHGNYYYYSGMDVWGQGTTVTVSSGGGGSGGGGSGGGGISYVLTQPPAVSGTPGQRVTISCSGSDSNIGRRSVNWYQQFPGTAPKLLIYSNDQRPSVVPDRFSGSKSGTSASLAISGLQSEDEAEYYCAAWDDSLKGAVFGGGTQLTV

SEQ ID NO: 10 - protein, 478 aa, Artificial Sequence
  Synthetic Polynucleotide
QVQLVQSGAEVKKPGSSVKVSCKSSGGTSNNYAISWVRQAPGQGLDWMGGISPIFGSTAYAQKFQGRVTISADIFSNTAYMELNSLTSEDTAVYFCARHGNYYYYSGMDVWGQGTTVTVSSGGGGSGGGGSGGGGISYVLTQPPAVSGTPGQRVTISCSGSDSNIGRRSVNWYQQFPGTAPKLLIYSNDQRPSVVPDRFSGSKSGTSASLAISGLQSEDEAEYYCAAWDDSLKGAVFGGGTQLTVEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRVPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 11 - protein, 593 aa, Artificial Sequence
  Synthetic Polynucleotide
QVQLQQSGVELVRPGTSVKMSCKASGYTFTNYWIGWAKQRPGHGLEWIGDIYPGGDYTNYNEKFRGKATLTADKSSSTAYMQFSSLTSEDSAIYYCASLYDGGFAYWGQGTLVTVSGGGGSGGGGSGGGGIQIVLTQSPAIMSASPGEKVTISCSASSSVSYMYWYQQKPGSSPKPWIYRTSNLASGVPARFSGSGSGTSYSLTISSMEAEDAATYYCQQFHGFPLTFGAGTKLELKEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRVPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 12 - protein, 9 aa, Artificial Sequence
  Synthetic Polynucleotide
IVLTQSPAI

SEQ ID NO: 13 - protein, 9 aa, Artificial Sequence
  Synthetic Polynucleotide
VLTQSPAIM

SEQ ID NO: 14 - protein, 9 aa, Artificial Sequence
  Synthetic Polynucleotide
VTISCSASS

SEQ ID NO: 15 - protein, 9 aa, Artificial Sequence
  Synthetic Polynucleotide
YWYQQKPGS

SEQ ID NO: 16 - protein, 9 aa, Artificial Sequence
  Synthetic Polynucleotide
WIYRTSNLA

SEQ ID NO: 17 - protein, 9 aa, Artificial Sequence
  Synthetic Polynucleotide
IYRTSNLAS

SEQ ID NO: 18 - protein, 9 aa, Artificial Sequence
  Synthetic Polynucleotide
LVRPGTSVK

SEQ ID NO: 19 - protein, 9 aa, Artificial Sequence
  Synthetic Polynucleotide
VKMSCKASG

SEQ ID NO: 20 - protein, 9 aa, Artificial Sequence
  Synthetic Polynucleotide
VRPGTSVKM

SEQ ID NO: 21 - protein, 9 aa, Artificial Sequence
  Synthetic Polynucleotide
FRGKATLTA

SEQ ID NO: 22 - protein, 9 aa, Artificial Sequence
  Synthetic Polynucleotide
YMQFSSLTS

SEQ ID NO: 23 - protein, 564 aa, Artificial Sequence
  Synthetic Polypeptide
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLINKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 24 - protein, 560 aa, Artificial Sequence
  Synthetic Polypeptide
MNTQILVFALIAIIPANADKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCHHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 25 - protein, 565 aa, Artificial Sequence
  Synthetic Polypeptide
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCR